(19) United States
(12) Patent Application Publication
(10) Pub. No.: US 2002/0026675 A1
(43) Pub. Date: Mar. 7, 2002

(54) Compositions for oxidation dyeing keratinous fibers comprising at least one 1-(4-aminophenyl)pyrrolidine and at least one thickening polymer comprising at least one sugar unit, and dyeing methods

(76) Inventors: Sylvain Kravtchenko, Asnieres (FR); Alain Lagrange, Coupvray (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 09/836,643

(22) Filed: Apr. 18, 2001

(30) Foreign Application Priority Data
Apr. 18, 2000 (FR) 00 04992

(51) Int. Cl. A61K 7/13

(52) U.S. Cl. 8/405; 8/406; 8/421

(57) ABSTRACT

Compositions for oxidation dyeing keratinous fibers, for example, human keratinous fibers such as hair, comprising, in a medium suitable for dyeing, at least one oxidation dye precursor chosen from 1-(4-aminophenyl)-pyrrolidines and acid addition salts thereof, and at least one thickening polymer comprising at least one sugar unit. Dyeing methods and devices using said compositions.

The present invention relates to compositions for oxidation dyeing keratinous fibers, for example, human keratinous fibers such as hair, comprising, in a medium suitable for dyeing, at least one oxidation dye precursor chosen from 1-(4-aminophenyl)-pyrrolidines and acid addition salts thereof, and at least one thickening polymer comprising at least one sugar unit.

It is known to dye keratinous fibers, for example, human hair, with dyeing compositions comprising oxidation dye precursors, generally called “oxidation bases.” Representative oxidation bases include ortho- and para-phenylenediamines, ortho- and para-aminophenols, and heterocyclic bases.

Oxidation dye precursors are compounds initially only slightly colored or not colored that develop their dyeing power in the hair in the presence of oxidizing agents, leading to the formation of colored compounds. The formation of these colored compounds results either from oxidative condensation of the “oxidation bases” with themselves, or oxidative condensation of the “oxidation bases” with color-modifying compounds, or “couplers,” which are generally present in the dyeing compositions used in oxidation dyeing. Representative couplers include meta-phenylenediamines, meta-aminophenols, meta-diphenols, and certain heterocyclic compounds.

The variety of compositions that can be employed in oxidation coloration, chosen from oxidation bases, oxidation couplers and mixtures of oxidation bases and couplers, can contribute to a palette very rich in color.

It is desirable for such oxidation dyes, otherwise called “permanent” dyes, to satisfy at least one of the following-make it possible to obtain shades of the desired intensity and tend to exhibit good resistance toward at least one external agent, such as, for example, light, adverse weather conditions, washing, permanent waving, perspiration, and rubbing.

Further, it is desirable that such dyes be able to cover grey hair, and thus should be the least selective possible, that is to say they should make it possible to obtain the smallest possible differences in color all along the same keratinous fiber, which may indeed be differently sensitized (i.e. damaged) between its tip and its root.

The permanent dyeing of hair with para-phenylenediamine (PPD) coupling products in the presence of hydrogen peroxide has been known for a long time and is in very widespread use. Nevertheless, better-tolerated oxidation bases have been sought and proposed as alternatives to PPD. For example, the tertiary base N,N-bis(β-hydroxyethyl)-para-phenylenediamine has been widely used in commercial hair-dyeing products, however, the colors obtained using these compositions tend to have at least one of the following drawbacks: less intense colors, less chromatic shades, or less resistant to various external agents to which the hair may be subjected.

The inventors have discovered that it is possible to overcome at least one of the above-mentioned drawbacks. By combining at least one (as used herein, “at least one” means one or more and thus includes mixtures and combinations) oxidation base chosen from 1-(4-aminophenyl)pyrrolidines and acid addition salts thereof with at least one thickening polymer comprising at least one sugar unit, however, the inventors have discovered that it is possible to obtain oxidation dyes capable of producing shades of colors that may have at least one of the following properties: shades that are varied, chromatic, intense, aesthetic, not very selective, and/or that exhibit good resistance to the various attacks to which the fibers may be subjected.

As used herein, the term “lower alkyl” means an alkyl chosen from saturated and unsaturated, branched and unbranched C₁-C₆ alkyl groups.

One subject of the invention is a composition for oxidation dyeing keratinous fibers, for example, human keratinous fibers such as hair, comprising, in an appropriate dyeing medium,

(i) at least one oxidation dye precursor chosen from 1-(4-aminophenyl)-pyrrolidines of formula (I) and acid addition salts thereof:

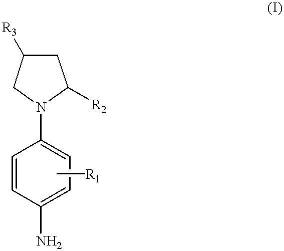

wherein:

R₁ is chosen from a hydrogen atom, C₁-C₆ alkyl groups, C₁-C₅ monohydroxyalkyl groups, and C₂-C₅ polyhydroxyalkyl groups,

R₂ is chosen from a hydrogen atom, a —CONH₂ group, C₁-C₅ monohydroxyalkyl groups, and C₂-C₅ polyhydroxyalkyl groups, and

R₃ is chosen from a hydrogen atom, and a hydroxyl group, and

(ii) at least one thickening polymer comprising at least one sugar unit.

The expression “sugar unit” is intended to mean, for the purposes of the present invention, a unit chosen from monosaccharide units, oligosaccharide units, and polysaccharide units. Representative monosaccharide units include, for example, monosaccharides, glycosides, and simple sugars. Representative oligosaccharide units are, for example, short chains formed of the linkage of monosaccharide units, which are optionally different. Representative polysaccharide units are long chains of monosaccharide units, which are optionally different, and can, for example, be chosen from homopolysaccharides and heteropolysaccharides. The saccharide units of said monosaccharide units, said oligosaccharide units, and said polysaccharide units may optionally be substituted with at least one group chosen from alkyl groups, hydroxyalkyl groups, alkoxy groups, acyloxy groups, and a carboxyl group.

The acid addition salts of said 1-(4-aminophenyl)pyrrolidines of formula (I) that can be used in the dyeing compositions according to the invention are chosen, for example, from hydrochlorides, hydrobromides, sulphates, tartrates, lactates, and acetates.

The dyeing compositions in accordance with the invention, after combining with an oxidizing composition comprising at least one oxidizing agent, tend to produce colors that exhibit at least one of the following properties: (1) varied, chromatic, intense, and/or aesthetic shades, (2) low selectivity, (3) resistance to atmospheric agents such as light and adverse weather conditions, and (4) resistance to perspiration and various treatments to which the hair may be subjected.

Another subject of the invention relates to a ready-to-use composition for oxidation dyeing keratinous fibers comprising, in an appropriate dyeing medium:

(i) at least one oxidation dye precursor chosen from 1-(4-aminophenyl)-pyrrolidines of formula (I) and acid addition salts thereof:

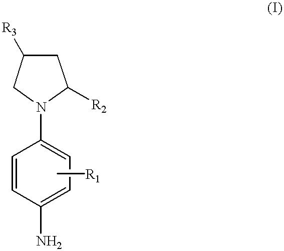

wherein:

R₁ is chosen from a hydrogen atom, C₁-C₆ alkyl groups, C₁-C₅ monohydroxyalkyl groups, and C₂-C₅ polyhydroxyalkyl groups,

R₂ is chosen from a hydrogen atom, a —CONH₂ group, C₁-C₅ monohydroxyalkyl groups, and C₂-C₅ polyhydroxyalkyl groups, and

R₃ is chosen from a hydrogen atom, and a hydroxyl group, and

(ii) at least one thickening polymer comprising at least one sugar unit, and

(iii) at least one oxidizing agent.

The term “ready-to-use composition” is understood to mean, for the purposes of the present invention, a composition intended to be applied immediately to the keratinous fibers, either stored as it is before use or obtained from the mixture of two or more compositions.

The invention also relates to a method for oxidation dyeing keratinous fibers, for example, human keratinous fibers such as hair, comprising:

(A) applying to said fibers at least one dyeing composition comprising, in a medium suitable for dyeing:

(i) at least one oxidation dye precursor chosen from 1-(4-aminophenyl)-pyrrolidines of formula (I) and acid addition salts thereof:

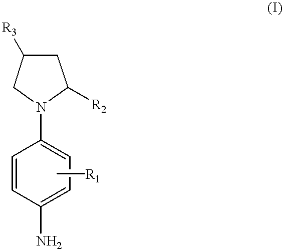

wherein:

R₁ is chosen from a hydrogen atom, C₁-C₆ alkyl groups, C₁-C₅ monohydroxyalkyl groups, and C₂-C₅ polyhydroxyalkyl groups, R₂ is chosen from a hydrogen atom, a —CONH₂ group, C₁-C₅ monohydroxyalkyl groups, and C₂-C₅ polyhydroxyalkyl groups, and

R₃ is chosen from a hydrogen atom, and a hydroxyl group,

and optionally comprising:

(ii) at least one thickening polymer comprising at least one sugar unit, and

(B) developing a color by applying to said fibers at least one oxidizing composition comprising:

at least one oxidizing agent and

optionally comprising said at least one thickening polymer comprising at least one sugar unit,

wherein said at least one oxidizing composition is

applied to said fibers after combining, at the time of use, said at least one oxidizing composition with said at least one dyeing composition, or

applied to said fibers either simultaneously with or immediately after said at least one dyeing composition, without intermediate rinsing, and

(C) provided that said at least one thickening polymer comprising at least one sugar unit is present in at least one of said at least one dyeing composition or said at least one oxidizing composition.

The invention also relates to a method for oxidation dyeing keratinous fibers, for example, human keratinous fibers such as hair, comprising:

(A) preparing, at the time of use, at least one dyeing composition comprising, in a medium suitable for dyeing:

(i) at least one oxidation dye precursor chosen from 1-(4-aminophenyl)-pyrrolidines of formula (I) and acid addition salts thereof:

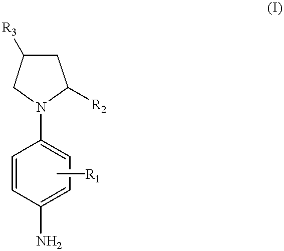

wherein:

R₁ is chosen from a hydrogen atom, C₁-C₆ alkyl groups, C₁-C₅ monohydroxyalkyl groups, and C₂-C₅ polyhydroxyalkyl groups,

R₂is chosen from a hydrogen atom, a —CONH₂ group C₁-C₅ monohydroxyalkyl groups, and C₂-C₅ polyhydroxyalkyl groups, and

R₃ is chosen from a hydrogen atom, and a hydroxyl group,

(ii) at least one thickening polymer comprising at least one sugar unit, and

(iii) at least one oxidizing agent,

(B) developing a color by applying said at least one dyeing composition prepared in (A) above to said fibers,

(C) leaving said at least one dyeing composition prepared in (A) above on said fibers for a time ranging, for example, from 1 to 60 minutes, such as, for example, from 10 to 45 minutes,

(D) rinsing said fibers, optionally shampooing said fibers, and optionally further rinsing said fibers, and

(E) drying said fibers.

According to the present invention, the hair that can be dyed in accordance with the method above can be chosen from, for example, wet hair and dry hair.

One embodiment of the invention relates to multicompartment dyeing devices or “kits” for oxidation dyeing keratinous fibers, for example, human keratinous fibers such as hair.

A kit for oxidation dyeing keratinous fibers according to the invention comprises at least two compartments, wherein:

(A) a first compartment comprises at least one dyeing composition comprising, in a medium suitable for dyeing:

(i) at least one oxidation dye precursor chosen from 1-(4-aminophenyl)-pyrrolidines of formula (I) and acid addition salts thereof:

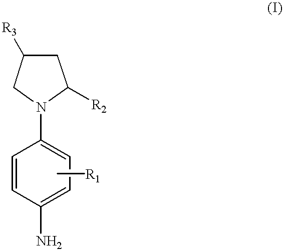

wherein:

R₁ is chosen from a hydrogen atom, C₁-C₆ alkyl groups, C₁-C₅ monohydroxyalkyl groups, and C₂-C₅ polyhydroxyalkyl groups,

R₂ is chosen from a hydrogen atom, a —CONH₂ group, C₁-C₅ monohydroxyalkyl groups, and C₂-C₅ polyhydroxyalkyl groups, and

R₃ is chosen from a hydrogen atom, and a hydroxyl group,

and optionally comprising:

(ii) at least one thickening polymer comprising at least one sugar unit,

(B) a second compartment comprises at least one oxidizing agent and optionally comprises said at least one thickening polymer comprising at least one sugar unit, and

(C) provided that said at least at least one thickening polymer comprising at least one sugar unit is present in at least one of said first compartment or said second compartment.

Another aspect of the invention relates to a kit for oxidation dyeing keratinous fibers comprising at least three compartments, wherein:

(A) a first compartment comprises at least one dyeing composition comprising, in a medium suitable for dyeing, at least one oxidation dye precursor chosen from 1-(4-aminophenyl)-pyrrolidines of formula (I) and acid addition salts thereof:

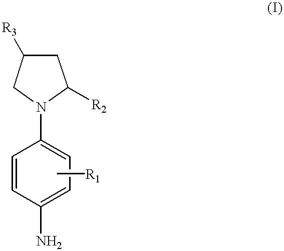

wherein:

R₁ is chosen from a hydrogen atom, C₁-C₆ alkyl groups, C₁-C₅ monohydroxyalkyl groups, and C₂-C₅ polyhydroxyalkyl groups,

R₂ is chosen from a hydrogen atom, a —CONH₂ group, C₁-C₅ monohydroxyalkyl groups, and C₂-C₅ polyhydroxyalkyl groups, and

R₃ is chosen from a hydrogen atom, and a hydroxyl group,

(B) a second compartment comprises at least one thickening polymer comprising at least one sugar unit, and

(C) a third compartment comprises at least one oxidizing agent.

Additional characteristics, aspects, objects, and advantages of the invention will emerge even more clearly on reading the description and examples which follow without however exhibiting a limiting character.

Representative 1-(4-aminophenyl)pyrrolidines of formula (I) according to the invention are, for example, described and prepared in U.S. Pat. Nos. 5,851,237, 5,876,464, and 5,993,491, the disclosures of which are incorporated by reference herein.

Representative 1-(4-aminophenyl)-pyrrolidines of formula (I) that can be used according to the invention can be chosen from such compounds wherein:

R₁, R₂, and R₃ are each a hydrogen atom, wherein said compound of formula (I) is 1-(4-aminophenyl)pyrrolidine,

R₁ and R₃ are each a hydrogen atom and R₂ is a -CH₂OH group, wherein said compound of formula (I) is 1-(4-aminophenyl)-2-pyrrolidinemethanol,

R₁ is a hydrogen atom, R₂ is a —CH₂OH group and R₃ is a hydroxyl group, wherein said compound of formula (I) is 1-(4-aminophenyl)-4-hydroxy-2-pyrrolidinemethanol, and

R₁ and R₃ are each a hydrogen atom and R₂ is a —CONH₂ group, wherein said compound of formula (I) is N-(4-aminophenyl)prolineamide.

The 1-(4-aminophenyl)pyrrolidines of formula (I) and acid addition salts thereof used in accordance with the invention can be present in the composition in an amount ranging, for example, from 0.001% to 10% by weight relative to the total weight of the composition, such as, for example, from 0.01% to 8% by weight relative to the total weight of the composition.

According to the present invention, the at least one thickening polymer comprising at least one sugar unit is chosen from:

(1) nonionic guar gums;

(2) biopolysaccharide gums of microbial origin, such as, for example, Scleroglucan gum and Xanthan gum;

(3) gums derived from plant exudates, such as, for example, gum Arabic, Ghatti gum, Karaya gum, Tragacanth gum, Carrageenan gum, Agar gum, and Carob gum;

(4) pectins;

(5) alginates;

(6) starches; and

(7) hydroxy(C₁-C₆)alkyl celluloses and carboxy(C₁-C₆)alkyl celluloses.

The nonionic guar gums are, for example, chosen from modified nonionic guar gums and unmodified nonionic guar gums.

The unmodified nonionic guar gums are, for example, the products sold under the name VIDOGUM GH 175 by the company UNIPECTINE, and under the names MEYPRO-GUAR 50 and JAGUAR C by the company MEYHALL.

The modified nonionic guar gums are, for example, nonionic guar gums that are modified by at least one (C₁-C₆) hydroxyalkyl group.

The at least one (C₁-C₆) hydroxyalkyl group can be chosen, for example, from a hydroxymethyl group, a hydroxyethyl group, a hydroxypropyl group, and a hydroxybutyl group.

Such nonionic guar gums are well known in the state of the art and may, for example, be prepared by reacting at least one alkene oxide with at least one nonionic guar gum. For example, propylene oxide can be reacted with a nonionic guar gum to obtain a nonionic guar gum modified by at least one hydroxypropyl group.

The degree of hydroxyalkylation, which corresponds to the number of alkylene oxide molecules consumed by the number of free hydroxyl functional groups present on the guar gum, can range, for example, from 0.4 to 1.2.

Such nonionic guar gums, optionally modified by hydroxyalkyl groups, are, for example, sold under the trade names JAGUAR HP8, JAGUAR HP60, JAGUAR HP120, JAGUAR DC₂₉₃, and JAGUAR HP 105 by the company Rhone Poulenc (MEYHALL) and under the name GALACTASOL 4H4FD2 by the company AQUALON.

The biopolysaccharide gums of microbial origin, such as, for example, scleroglucan gum, xanthan gum, the gums derived from plant exudates, such as, for example, gum Arabic, Ghatti gum, Karaya gum, Tragacanth gum, Carrageenan gum, Agar gum, and Carob gum, said hydroxyalkyl celluloses, said carboxymethyl celluloses, said pectins, said alginates, and said starches are well known to persons skilled in the art and are described, for example, in the book by Robert L. Davidson entitled “Handbook of Water Soluble Gums and Resins” published by McGraw Hill Book Company (1980), the disclosure of which is incorporated by reference herein.

Among such gums, said scleroglucan gums that can be used according to the present invention are, for example, chosen from the products sold under the name ACTIGUM CS by the company SANOFI BIO INDUSTRIES, such as, for example, ACTIGUM CS 11, and the products sold under the name AMIGEL by the company ALBAN MULLER INTERNATIONAL. Other scleroglucan gums that may also be used include, for example, scleroglucan gums treated with glyoxal, which are described, for example, in French Patent Application No. 2,633,940, the disclosure of which is incorporated by reference herein.

The Xanthan gums that may be used according to the present invention include, for example, the products sold under the names KELTROL, KELTROL T, KELTROL TF, KELTROL BT, KELTROL RD, and KELTROL CG by the company NUTRASWEET KELCO, and the products sold under the names RHODICARE S and RHODICARE H by the company RHODIA CHIMIE.

The hydroxy(C₁-C₆)alkyl celluloses can be chosen, for example, from hydroxyethyl celluloses, such as, for example, the products sold under the names CELLOSIZE QP3L, CELLOSIZE QP4400H, CELLOSIZE QP30000H, CELLOSIZE HEC30000A, and CELLOSIZE POLYMER PCG10 by the company AMERCHOL, the products sold under the names NATROSOL 250HHR, NATROSOL 250MR, NATROSOL 250M, NATROSOL 250HHXR, NATROSOL 250HHX, NATROSOL 250HR, and NATROSOL HX by the company HERCULES, and the products sold under the name TYLOSE H1000 by the company HOECHST.

The hydroxy(C₁-C₆)alkyl celluloses can also be chosen from, for example, the products sold under the names KLUCEL EF, KLUCEL H, KLUCEL LHF, KLUCEL MF, and KLUCEL G by the company AQUALON.

Representative carboxy(C₁-C₆)alkyl celluloses include, for example, carboxymethyl celluloses, which are, for example, sold as the products BLANOSE 7M8/SF, BLANOSE RAFFINEE 7M, BLANOSE 7LF, BLANOSE 7MF, BLANOSE 9M31 F, BLANOSE 12M31XP, BLANOSE 12M31P, BLANOSE 9M31XF, BLANOSE 7H, BLANOSE 7M31, and BLANOSE 7H3SXF by the company AQUALON, the products AQUASORB A500 and AMBERGUM 1221 by the company HERCULES, the products CELLOGEN HP810A and CELLOGEN HP6HS9 by the company MONTELLO, and the products PRIMELLOSE by the company AVEBE.

The at least one thickening polymer comprising at least one sugar unit can be present in the composition of the invention in an amount ranging from, for example, 0.01% to 10% by weight relative to the total weight of said composition, such as, for example, from 0.1% to 5% by weight relative to the total weight of said composition.

The compositions according to the invention can also comprise at least one coupler. Representatives of the at least one coupler can include, for example, meta-phenylenediamines, meta-aminophenols, meta-diphenols, naphthols, and heterocyclic couplers, such as, for example, indole derivatives, indoline derivatives, sesamol and its derivatives, pyridine derivatives, pyrazolotriazole derivatives, pyrazolones, indazoles, benzimidazoles, benzothiazoles, benzoxazoles, 1,3-benzodioxoles, quinolines, and acid addition salts of any of the foregoing compounds.

Representatives of the at least one coupler can include, for example, couplers chosen from 2,4-diamino-1-(β-hydroxyethyloxy)benzene, 2-methyl-5-aminophenol, 5-N-(p-hydroxyethyl) amino-2-methylphenol, 3-aminophenol, 1,3-dihydroxybenzene, 1,3-dihydroxy-2-methylbenzene, 4-chloro-1,3-dihydroxy-benzene, 2-amino-4-(β-hydroxyethylamino)-1-methoxy-benzene, 1,3-diaminobenzene, 1,3-bis(2,4-diamino-phenoxy)propane, sesamol, 1-amino-2-methoxy-4,5-methylenedioxybenzene, α-naphthol, 6-hydroxyindole, 4-hydroxyindole, 4-hydroxy-N-methylindole, 6-hydroxy-indoline, 2,6-dihydroxy-4-methylpyrid ine, 1-H-3-methyl-pyrazol-5-one, 1-phenyl-3-methylpyrazol-5-one, 2-amino-3-hydroxypyrid ine, 3,6-d imethylpyrazolo[3,2-c]-1,2,4-triazole, 2,6-dimethylpyrazolo[1,5-b]-1,2,4-triazole and acid addition salts of any of the foregoing compounds.

The at least one coupler can be present in the composition according to the invention in an amount ranging, for example, from 0.0001% to 15% by weight relative to the total weight of the composition.

The dyeing compositions in accordance with the invention may further comprise at least one direct dye. Further, the dyeing compositions in accordance with the invention may further comprise at least one additional oxidation base chosen from oxidation bases other than said 1-(4-aminophenyl)pyrrolidines of formula (I) and acid addition salts thereof.

Representatives of the at least one additional oxidation bases which can be used according to the invention, include, for example, para-phenylenediamine, para-tolylenediamine, 2-hydroxyethyl-para-phenylenediamine, 1-N,N-bis(2-hydroxyethyl)-para-phenylenediamine, para-aminophenols such as 3-methyl-4-aminophenol and 4-aminophenol, ortho-phenylenediamines, ortho-aminophenols, double bases, and heterocyclic bases, such as pyrimidines, for example, 2,4,5,6-tetraaminopyrimidine, and pyrazoles, for example, 1-(2-hydroxyethyl)-4,5-diaminopyrazole, and acid addition salts of any of the foregoing compounds.

Said at least one additional oxidation base may be present in the composition according to the invention in an amount ranging, for example, from 0.0001% to 15% by weight relative to the total weight of said composition.

One embodiment of the dyeing composition in accordance with the invention may, in addition, comprise at least one direct dye, for example, to modify the shades of the dyes by enriching them with glints. The at least one direct dye may be chosen from neutral, cationic, and anionic nitro dyes; neutral, cationic, and anionic azo dyes; and neutral, cationic, and anionic anthraquinone dyes.

The at least one direct dye can be present in the composition according to the invention in an amount ranging, for example, from 0.001% to 20% by weight relative to the total weight of said composition, such as, for example, from 0.01% to 10% by weight relative to the total weight of the composition.

The medium suitable for dyeing according to the invention can be, for example, an aqueous medium comprising water and may further comprise at least one cosmetically acceptable organic solvent. The at least one cosmetically acceptable organic solvent may, for example, be chosen from alcohols, such as ethyl alcohol, isopropyl alcohol, benzyl alcohol, and phenylethyl alcohol; glycols (for example, ethyleneglycol, propyleneglycol, butyleneglycol, dipropyleneglycol, and diethyleneglycol); and ethers of glycols (for example, monomethyl, monoethyl and monobutyl ethers of ethyleneglycol and for example monomethyl ether of propyleneglycol and alkyl ethers of diethyleneglycol glycol, such as, for example, monoethylether and monobutylether of diethyleneglycol). The at least one cosmetically acceptable organic solvent may be present in the composition according to the invention in an amount ranging, for example, from 0.5% to 20% by weight relative to the total weight of the composition, such as, for example, from 2% to 10% by weight relative to the total weight of the composition.

The composition according to the invention may further comprise an effective quantity of other agents. For example, agents that are already known in oxidation dyeing, such as various customary adjuvants such as sequestrants such as EDTA and etidronic acid, UV-screening agents, waxes, volatile and nonvolatile, cyclic and linear, branched and unbranched silicones, preservatives, ceramides, pseudoceramides, vegetable, mineral, and synthetic oils, vitamins and provitamins such as panthenol, opacifiers, thickening agents other than those defined according to the present invention, as well as cationic polymers, may be added.

Cationic polymers

As used herein, “cationic polymer” refers to polymers chosen from polymers comprising at least one cationic group and polymers comprising at least one group which can be ionized to form cationic groups.

Representative cationic polymers which may be used in accordance with the present invention include any of those already known to improve at least one cosmetic property of hair, such as, for example, those described in patent application EP-A-0 337 354 and in French patent applications FR-A-2 270 846, 2 383 660, 2 598 611, 2 470 596 and 2 519 863, the disclosures of which are incorporated herein by reference.

According to the present invention, the at least one cationic polymer may be chosen from polymers comprising at least one unit, wherein said at least one unit comprises at least one group chosen from primary amine groups, secondary amine groups, tertiary amine groups and quaternary amine groups, wherein said at least one group forms part of the polymer skeleton, or is carried by at least one lateral substituent on said polymer skeleton.

According to the present invention, the at least one cationic polymer has a number-average molecular mass generally ranging, for example, from 500 to 5×10⁶, such as, for example, from 1×10³ to 3×10⁶.

The at least one cationic polymer may, for example, be chosen from polymers of quaternary polyammonium type, polymers of polyamino amide type and polymers of polyamine type. Such types of polymers are known in the art. They are for example described in French patents Nos. 2,505,348 and 2,542,997, the disclosures of which are incorporated by reference herein.

Non-limiting examples of cationic polymers include:

(1) homo- and co-polymers derived from at least one monomer chosen from acrylic esters, methacrylic esters and amides, wherein said homo- and co-polymers comprise at least one unit chosen from units of formulae (1), (11), (111), and (IV):

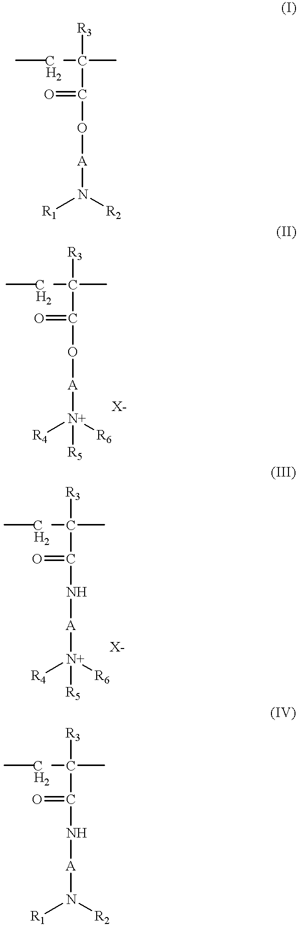

wherein:

R₃, which may be identical or different, are each chosen from a hydrogen atom and a methyl group,

A, which may be identical or different, are each chosen from linear and branched divalent (C₁-C₆) alkyl groups, such as (C₂-C₃) alkyl groups, and (C₁-C₄) hydroxyalkyl groups,

R₄, R₅ and R₆, which may be identical or different, are each chosen from (C₁-C₁₈) alkyl groups, such as (C_{1-C}₆) alkyl groups, and benzyl groups;

R₁ and R₂, which may be identical or different, are each chosen from a hydrogen atom and (C₁-C₆) alkyl groups, such as a methyl group and an ethyl group,

X⁻ is an anion chosen from anions derived from at least one inorganic acid and anions derived from at least one organic acid, such as a methosulfate anion and halides, such as a chloride anion and a bromide anion.

Copolymers of family (1) may further comprise at least one unit derived from at least one comonomer chosen from vinyllactams, vinyl esters, acrylamides, methacrylamides, diacetone acrylamides, acrylamides and methacrylamides substituted on the nitrogen with at least one group chosen from (C₁-C₄) alkyls, acrylic acid, methacrylic acid, acrylic acid esters, and methacrylic acid esters. Non-limiting examples of vinyllactams include vinylpyrrolidone and vinylcaprolactam.

Non-limiting examples of copolymers of family (1) include:

copolymers derived from at least one monomer of (i) acrylamide and (ii) dimethylaminoethyl methacrylate quaternized with at least one group chosen from dimethyl sulfate and dimethyl halide, such as, for example, the product sold under the name HERCOFLOC by the company Hercules;

copolymers derived from at least one monomer of (i) acrylamide and (ii) methacryloyloxy-ethyltrimethylammonium chloride described, for example, in patent application EP-A-080 976, the disclosure of which is incorporated herein by reference, and which is sold, for example, under the name BINA QUAT P 100 by the company Ciba Geigy;

copolymers derived from at least one monomer of (i) acrylamide and (ii) methacryloyloxy-ethyltrimethylammonium methosulfate, such as, for example, copolymers sold under the name RETEN by the company Hercules;

quaternized and non-quaternized vinylpyrrolidone/dialkylaminoalkyl acrylate copolymers and quaternized and non-quaternized vinylpyrrolidone/dialkylaminoalkyl methacrylate copolymers, such as, for example, the products sold under the name “GAFQUAT” by the company ISP, such as, for example, “GAFQUAT 734” or “GAFQUAT 755” and the products known as “COPOLYMER 845, 958 and 937”.

These polymers are described in detail in French patents 2,077,143 and 2,393,573, the disclosures of which are incorporated herein by reference;

dimethylaminoethyl methacrylate/vinylcaprolactam/vinylpyrrolidone terpolymers, such as, for example, the product sold under the name GAFFIX VC₇₁₃ by the company ISP;

vinylpyrrolidone/methacrylamidopropyldimethylamine copolymers, such as, for example, the product sold under the name STYLEZE CC₁₀ by ISP; and

quaternized vinylpyrrolidone/dimethylaminopropylmethacrylamide copolymers, such as, for example, the product sold under the name “GAFQUAT HS 100” by the company ISP.

Further, non-limiting examples of cationic polymers include:

(2) cellulose ether derivatives comprising quaternary ammonium groups, such as, for example, those described in French patent 1,492,597, the disclosure of which is incorporated herein by reference, and polymers sold under the names “JR” (JR 400, JR 125 and JR 30M) and “LR” (LR 400, or LR 30M) by the company Union Carbide Corporation. These polymers are also defined in the CTFA dictionary as quaternary ammoniums of hydroxyethylcellulose which have reacted with an epoxide substituted with a trimethylammonium group;

(3) cationic cellulose derivatives such as cellulose copolymers and cellulose derivatives grafted with at least one water-soluble monomer of quaternary ammonium, such as, for example, those described in U.S. Pat. No. 4,131,576, the disclosure of which is incorporated herein by reference, such as hydroxyalkylcelluloses (such as, for example, hydroxymethylcelluloses, hyd roxyethylcellu loses and hydroxypropylcelluloses, wherein said hydroxyalkylcelluloses are grafted with at least one salt chosen from, for example, methacryloylethyltrimethylammonium salts, methacrylamidopropyltrimethylammonium salts and dimethyidiallylammonium salts). For example, commercial products corresponding to the aforementioned cationic cellulose derivatives include, for example, the products sold under the names “CELQUAT L 200” and “CELQUAT H 100” by the company National Starch;

(4) cationic polysaccharides, such as, for example, those described in U.S. Pat. Nos. 3,589,578 and 4,031,307, the disclosures of which are incorporated herein by reference, such as guar gums comprising at least one cationic trialkylammonium group. For example, guar gums modified with at least one salt, such as a chloride salt, of 2,3-epoxypropyltrimethylammonium may be used in the present invention. Such products are sold, for example, under the trade names JAGUAR C13 S, JAGUAR C₁₅, JAGUAR C₁₇ and JAGUAR C162 by the company Meyhall;

(5) polymers comprising (i) at least one piperazinyl unit and (ii) at least one group chosen from divalent alkylene groups and divalent hydroxyalkylene groups, wherein said at least one group optionally comprises at least one chain chosen from straight chains and branched chains, wherein said at least one chain is optionally interrupted by at least one entity chosen from an oxygen atom, a sulfur atom, a nitrogen atom, aromatic rings, and heterocyclic rings, the oxidation products of said polymers and the quaternization products of said polymers. For example, such polymers are described in French patents 2,162,025 and 2,280,361, the disclosures of which are incorporated herein by reference;

(6) water-soluble polyamino amides which may be prepared by at least one polycondensation reaction of at least one acidic compound and at least one polyamine compound, wherein said polyamino amides may be crosslinked with at least one crosslinking agent chosen from epihalohydrins, diepoxides, dianhydrides, unsaturated dianhydrides, bis-unsaturated derivatives, bis-halohydrins, bis-azetidiniums, bis-haloacyldiamines, bis-alkyl halides and oligomers derived from reaction of at least one difunctional compound with at least one compound chosen from bis-halohydrins, bis-azetidiniums, bis-haloacyidiamines, bis-alkyl halides, epihalohydrins, diepoxides and bis-unsaturated derivatives, wherein said crosslinking agent may be used in a proportion generally ranging, for example, from 0.025 mol to 0.35 mol per amine group of said polyamino amide, wherein said polyamino amides may optionally be alkylated, and wherein, if said polyamino amides comprise at least one tertiary amine group, said polyamino amides may optionally be quaternized. For example, such polymers are, for example, described in French patents 2,252,840 and 2,368,508, the disclosures of which are incorporated by reference herein; and

(7) polyamino amide derivatives derived from condensation of at least one polyalkylene polyamine with at least one polycarboxylic acid, followed by alkylation with at least one bifunctional agent. Non-limiting examples of such polyamino amide derivatives include adipic acid/dialkylaminohydroxyalkyldialkylenetriamine polymers, wherein the alkyl group is chosen form (C₁-C₄) alkyl groups, such as, for example, a methyl group, an ethyl group, and a propyl group. For example, such polymers are described in French patent 1,583,363, the disclosure of which is incorporated herein by reference.

Other non-limiting examples of such derivatives include the adipic acid/dimethylaminohydroxypropyl/diethylenetriamine polymers sold, for example, under the name “CARTARETINE F, F4 or F8” by the company Sandoz.

Further, non-limiting examples of cationic polymers include:

(8) polymers derived from the reaction of (i) at least one polyalkylene polyamine comprising two primary amine groups and at least one secondary amine group with (ii) at least one dicarboxylic acid chosen from diglycolic acid and saturated aliphatic dicarboxylic acids comprising from 3 to 8 carbon atoms. According to the present invention, the molar ratio of the at least one polyalkylene polyamine to the at least one dicarboxylic acid generally ranges, for example, from 0.8:1 to 1.4:1. The polyamino amide resulting from the above reaction may be reacted with epichlorohydrin in a molar ratio of epichlorohydrin to the at least one secondary amine group of the polyamino amide generally ranging, for example, from 0.5:1 to 1.8:1. For example, such polymers are described in U.S. Pat. Nos. 3,227,615 and 2,961,347, the disclosures of which are incorporated herein by reference.

Polymers of this type are sold, for example, under the name “HERCOSETT 57” by the company Hercules Inc. and, further, for example, under the names “PD 170” and “DELSETTE 101” by the company Hercules in the case of adipic acid/epoxypropyl/diethylenetriamine copolymers.

Further, non-limiting examples of cationic polymers include:

(9) cyclopolymers of alkyldiallylamine and cyclopolymers of dialkyldiallylammonium, such as homopolymers and copolymers comprising, as a constituent of the chain, at least one unit chosen from units of formulae (V) and (VI):

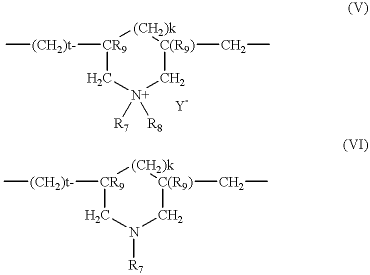

wherein:

k and t, which are identical or different, are each chosen from 0 and 1, provided that the sum of k+t is equal to 1,

R₇ and R₈, which are identical or different, are each chosen from (C₁-C₂₂) alkyl groups, such as, for example, (C₁-C₄) alkyl groups, hydroxyalkyl groups, such as, for example, (C₁-C₅)-hydroxyalkyl groups, and (C₁-C₄)amidoalkyl groups,

R₇and R₈, together with the nitrogen cation to which they are commonly bonded, may optionally form a cationic heterocyclic group chosen from a piperidinyl group and a morpholinyl group,

R₉, which are identical or different, are each chosen from a hydrogen atom and a methyl group, and

Y⁻ is an anion, such as, for example, an anion chosen from a bromide anion, a chloride anion, an acetate anion, a borate anion, a citrate anion, a tartrate anion, a bisulfate anion, a bisulfite anion, a sulfate anion, and a phosphate anion. For example, such polymers are described in French patent 2,080,759 and in its Certificate of Addition 2,190,406, the disclosures of which are incorporated herein by reference.

The cationic polymers comprising repeating units chosen from repeating units of formula (V) and (VI) can include, for example, the homopolymer of dimethyidiallylammonium chloride sold under the name “Merquat 100” by the company Calgon (and its homologues of low weight-average molecular mass) and the copolymers of dimethyidiallylammonium chloride and of acrylamide marketed under the name “MERQUAT 550”.

Further, non-limiting examples of cationic polymers include:

(10) quaternary diammonium polymers comprising repeating units of formula (VII):

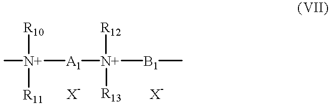

wherein:

R₁₀, R₁₁, R₁₂, and R₁₃, which are identical or different, are each chosen from C₁-C₂₀ aliphatic groups, C₃-C₂₀ alicyclic groups, C₇-C₂₀ arylaliphatic groups, and lower hydroxyalkyl groups,

at least two of said R₁₀, said R₁₁, said R₁₂, and said R₁₃, together with the nitrogen cations to which they are attached, optionally form at least one cationic heterocyclic ring optionally comprising an additional heteroatom other than nitrogen,

R₁₀, R₁₁, R₁₂, and R₁₃, which are identical or different, optionally are each chosen from linear and branched C₁-C₆ alkyl groups substituted with at least one group chosen from nitrile groups, ester groups, acyl groups, amide groups, —CO—O—R₁₄—D groups, and —CO—NH—R₁₄—D groups, wherein R₁₄ is chosen from alkylene groups and D is chosen from quaternary ammonium groups,

A₁, and B₁, which are identical or different, are each chosen from linear and branched, saturated and unsaturated, C₂-C₂₀ polymethylene groups, optionally comprising at least one entity chosen from aromatic rings, an oxygen atom, a sulfur atom, a sulfoxide group, a sulfone group, a disulfide group, an amino group, alkylamino groups, a hydroxyl group, quaternary ammonium groups, a ureido group, an amide group, and ester groups, wherein said at least one entity is linked to or intercalated in the main chain,

X⁻ is an anion chosen from anions derived from inorganic acids and organic acids, such as, for example, chosen from a chloride anion and a bromide anion,

said A₁, said R₁₀, and said R₁₂ optionally form a piperazine ring, together with the two nitrogen cations to which they are attached, and

provided that if A₁ is chosen from linear and branched, saturated and unsaturated, C₂-C₂₀ polymethylene groups and linear and branched, saturated and unsaturated, hydroxy(C₂-C₂₀)polymethylene groups, B₁ is additionally chosen from —(CH₂)ₙ—CO—D—OC—(CH₂)ₙ— groups, wherein:

n is an integer ranging from 1 to 100, such as, for example, from 1 to 50, and

D is chosen from:

a) glycol residues of formula: —O—Z—O—, wherein Z is chosen from linear and branched hydrocarbon groups and groups chosen from groups of formulae:

—(CH₂—CH₂—O)ₓ—CH₂—CH₂— and

—[CH₂—CH(CH₃)—O]_{y}—CH₂—CH(CH₃)—

wherein x and y, which are identical or different, are each chosen from integers ranging from 1 to 4 (in which case x and y represent a defined and unique degree of polymerization) and any number ranging from 1 to 4 (in which case x and y represent an average degree of polymerization),

b) bis-secondary diamine residues such as piperazine derivatives,

c) bis-primary diamine residues chosen from residues of formula: —NH—Y—NH—, wherein Y is chosen from linear and branched hydrocarbon groups and residues of formula —CH₂—CH₂—S—S—CH₂—CH₂—, and

d) a ureylene group of formula: —NH—CO—NH—.

The cationic polymers comprising repeating units of formula (VII) that can be used according to the invention, for example, can have a number-average molecular mass generally ranging, for example, from 1,000 to 100,000.

For example, the cationic polymers comprising repeating units of formula (VII) are described, for example, in French Patent Nos. 2,320,330, 2,270,846, 2,316,271, 2,336,434 and 2,413,907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020, the disclosures of which are incorporated herein by reference.

Further, representative quaternary diammonium polymers comprising repeating units of formula (VII) can, for example, be chosen from polymers comprising repeating units of formula (VIII):

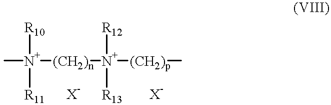

wherein

R₁₀, R₁₁, R₁₂, and R₁₃, which are identical or different, are each chosen from C₁-C₄ alkyl groups and C₁-C₄ hydroxyalkyl groups,

n and p are each chosen from integers ranging from 2 to 20, and

—X⁻ is an anion chosen from anions derived from inorganic acids and anions derived from organic acids.

Further, non-limiting examples of cationic polymers include:

(11) quaternary diammonium polymers comprising repeating units of formula (IX):

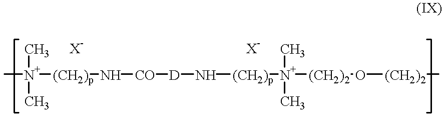

wherein:

p is an integer ranging from 1 to 6,

D is chosen from a direct bond and —(CH₂)ᵣ—CO— groups, wherein r is a number equal to 4 or 7, and

X⁻ is an anion.

Such cationic polymers comprising repeating units of formula (IX) may be prepared, for example, according to the methods described in U.S. Pat. Nos. 4,157,388, 4,390,689, 4,702,906, and 4,719,282, the disclosures of which are incorporated by reference herein.

In one embodiment of the invention, said cationic polymers comprising repeating units of formula (IX) are chosen from:

(a) repeating units of formula (IX), wherein p is equal to 3, D is a —(CH₂)₄—CO— group, X⁻ is a chloride anion, and the number-average molecular mass, measured by Carbon 13 NMR (¹³C NMR) is 5,600; a polymer of this type is provided by the company MIRANOL under the name MIRAPOL-AD1,

(b) repeating units of formula (IX), wherein p is equal to 3, D is a —(CH₂)₇—CO— group, X⁻0 is a chloride anion, and the number-average molecular mass, measured by Carbon 13 NMR (¹³C NMR) is 8,100; a polymer of this type is provided by the company MIRANOL under the name MIRAPOL-AZI,

(c) repeating units of formula (IX), wherein p is equal to 3, D is a direct bond, X⁻ is a chloride anion, and the number-average molecular mass, measured by Carbon 13 NMR (¹³C NMR) is 25,500; a polymer of this type is sold by the company MIRANOL under the name MIRAPOL-A15, and

(d) repeating units of formula (IX) described in sections (a) and (c) above, wherein a “Block Copolymer” is formed; polymers of this type include polymers provided by the company MIRANOL, under the names MIRAPOL-9, (wherein the number-average molecular mass, measured by Carbon 13 NMR (¹³C NMR) is 7,800) MIRAPOL-175, (wherein the number-average molecular mass, measured by Carbon 13 NMR (¹³C NMR) is 8,000), and MIRAPOL-95 (wherein the number-average molecular mass, measured by Carbon 13 NMR (¹³C NMR) is 12,500).

Further, non-limiting examples of cationic polymers include:

(12) quaternary polymers of vinylpyrrolidone and quaternary polymers of vinylimidazole, such as, for example, the products sold under the names LUVIQUAT FC₉₀₅, FC₅₅₀ and FC₃₇₀ by the company BASF;

(13) polyamines, such as, for example, POLYQUART H sold by Henkel under the reference name “POLYETHYLENE GLYCOL (15) TALLOW POLYAMINE” in the CTFA dictionary; and

(14) crosslinked (meth)acryloyloxy(C₁-C₄)alkyltri(C₁-C₄)alkylammonium salt polymers, such as the polymers derived from homopolymerization of dimethylaminoethyl methacrylate quaternized with methyl chloride and polymers derived from copolymerization, for example, of acrylamide with dimethylaminoethyl methacrylate quaternized with a methyl halide (such as methyl chloride), wherein the homo- or copolymerization is followed by crosslinking with at least one compound comprising olefinic unsaturation, such as methylenebisacrylamide. For example, a crosslinked acrylamide/methacryloyloxyethyltrimethylammonium chloride copolymer (20/80 by weight) in the form of a dispersion comprising about 50% by weight of said copolymer in mineral oil may be used. This dispersion is sold under the name “SALCARE SC₉₂” by the company Allied Colloids. Further, a crosslinked methacryloyloxyethyltrimethylammonium chloride homopolymer comprising about 50% by weight of the homopolymer in mineral oil or in a liquid ester may be used. These dispersions are, for example, sold under the names “SALCARE SC₉₅” and “SALCARE SC₉₆” by the company Allied Colloids.

Other cationic polymers which may be used as the at least one cationic polymer according to the present invention are polyalkyleneimines (such as polyethyleneimines), polymers comprising at least one vinylpyridine unit, polymers comprising at least one vinylpyridinium unit, condensates of polyamines, condensates of epichlorohydrin, quaternary polyureylenes and chitin derivatives.

Other embodiments of the invention use cationic polymers chosen from the polymers of (1), (9), (10), (11) and (14) as defined above. Specifically, polymers of family (10) chosen from polymers comprising repeating units of formula (W) and polymers comprising repeating units of formula (U) can be used:

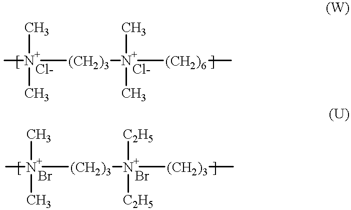

Said cationic polymers with (W) repeating units and said cationic polymers with (U) repeating units are prepared and described, for example, in French Patent 2,270,846, the disclosure of which is incorporated by reference herein.

The at least one cationic polymer can be present in the composition according to the invention in an amount ranging from, for example, 0.01% to 10% by weight relative to the total weight of the composition, such as, for example, from 0.05% to 5% by weight relative to the total weight of the composition, and further such as, for example, from 0.1% to 3% by weight relative to the total weight of the composition.

The compositions may also comprise at least one agent chosen from reducing agents and antioxidants. Representative agents may include, for example, sodium sulphite, thioglycolic acid, thiolactic acid, sodium bisulphite, dehydroascorbic acid, hydroquinone, 2-methylhydroquinone, tert-butylhydroquinone, and homogentisic acid. Such agents are generally present in the composition of the invention in an amount ranging from, for example, 0.05% to 1.5% by weight relative to the total weight of the composition.

The composition according to the invention may also comprise at least one fatty alcohol. Representatives of the at least one fatty alcohol that can be used according to the invention can include, for example, lauryl alcohol, cetyl alcohol, stearyl alcohol, and oleyl alcohol. The at least one fatty alcohol may be present in the composition according to the invention in an amount ranging, for example, from 0.001% to 20% by weight relative to the total weight of the composition.

The compositions according to the invention may further comprise at least one surfactant chosen from nonionic, anionic, cationic, and amphoteric surfactants. Said at least one surfactant may be present in the composition according to the invention in at least one of said at least one dyeing composition or said at least one oxidizing composition. Said at least one surfactant can be present in the composition according to the invention an amount ranging, for example, from 0.1% to 20% by weight relative to the total weight of said composition.

In one embodiment, the composition according to the invention comprises at least one nonionic surfactant.

One skilled in the art should take care to select said optionally complementary compounds, such that the advantageous properties intrinsically associated with the dye composition according to the invention are not, or are not substantially, adversely affected by the additions envisaged.

Said at least one oxidizing composition according to the invention comprises at least one oxidizing agent. Said at least one oxidizing agent can, for example, be chosen from hydrogen peroxide, urea peroxide, alkali metal bromates, alkali metal ferricyanides, and persalts such as perborates and persulphates. In one embodiment, said at least one oxidizing agent can, for example, be hydrogen peroxide. When said at least one oxidizing agent is hydrogen peroxide, said at least one oxidizing agent may, for example, comprise a solution of hydrogen peroxide with a titre ranging, for example, from 1 to 40 in volume, such as, for example, from 5 to 40 in volume.

It is also possible to use as said at least one oxidizing agent at least one oxidation-reduction enzyme. Said oxidation-reduction enzymes can be chosen from, for example, laccases, peroxidases, and 2-electron oxidoreductases (such as uricase), where appropriate in the presence of their respective donor or cofactor.

The pH of the dyeing composition or of the ready-to-use composition (composition resulting from combining said at least one dyeing composition with said at least one oxidizing composition) applied to the keratinous fibers, generally ranges, for example, from 3 to 12, such as, for example, from 6 to 11. Said pH may be adjusted to the desired value by means of at least one agent for adjusting said pH chosen from acidifying agents and alkalinizing agents well known in the state of the art for dyeing keratinous fibers.

Representative alkalinizing agents may be chosen from, for example, aqueous ammonia, alkali metal carbonates, alkanolamines such as mono-, di- and triethanolamines as well their derivatives, oxyethylenated ethylenediamines, oxypropylenated ethylenediamines, hydroxyalkylamines, sodium hydroxide, potassium hydroxide, and compounds of formula (XI):

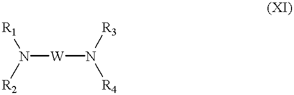

wherein:

W is a propylene residue optionally substituted with a group chosen from a hydroxyl group and C₁-C₄ alkyl groups;

R₁, R₂, R₃, and R₄, which are identical or different, are each chosen from hydrogen, (C₁-C₄) alkyl groups, and (C₁-C₄) hydroxyalkyl groups.

The acidifying agents are conventionally, by way of example, chosen from, inorganic acids, and organic acids, such as, for example, hydrochloric acid, orthophosphoric acid, carboxylic acids such as tartaric acid, citric acid, lactic acid, and sulphonic acids.

The dyeing composition in accordance with the invention may be provided in various forms, such as in the form of liquids, powders, creams, gels, optionally pressurized, or in any other form appropriate for dyeing keratinous fibers, and in particular human hair.

Concrete examples illustrating the invention are indicated below without however exhibiting a limiting character.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

(expressed in grams)

| | EXAMPLE 1 | EXAMPLE 2 |
|---|---|---|
| | invention | prior art |
| 1-(4-Aminophenyl)pyrrolidine | 0.470 | |
| dihydrochloride (oxidation base | | |
| according to the invention) | | |
| 2,4-Diamino-1-(β-hydroxyethyloxy)- | 0.482 | 0.482 |
| benzene dihydrochloride (coupler) | | |
| N,N-bis(β-Hydroxyethyl)-para- | | 0.392 |
| phenylenediamine sulphate (oxidation | | |
| base according to the prior art) | | |
| Nonionic guar gum (MEYPRO- | 0.75 | 0.75 |
| GUAR 50 from MEYHALL) | | |
| Dye carrier (*) | qs | qs |
| Demineralized water qs | 100 | 100 |

(*) Dye carrier composition

| | | |
|---|---|---|
| C₈—C₁₀ alkyl polyglucoside in aqueous solution at 60%, | 3.24 | g AS* |
| sold under the name ORAMIX CG 110 ® | | |
| by the company SEPPIC | | |
| Ethanol | 18.0 | g |
| Benzyl alcohol | 1.8 | g |
| Polyethylene glycol 400 | 2.7 | g |
| Pentasodium salt of diethylenetriaminepentaacetic acid | 0.43 | g AS* |
| in aqueous solution at 40%, sold under the name | | |
| DISSOLUINE D-40 ® by the company AKZO | | |
| Sodium metabisulphite | 0.205 | g |
| Aqueous ammonia containing 20.5% of NH₃ | 10.0 | g |

At the time of use, each of the dyeing compositions described above was combined, weight for weight, with a solution of hydrogen peroxide at 20 volumes (6% by weight).

The combinations thus prepared were applied for 30 minutes to locks of natural grey hair which is 90% white. The locks were then rinsed, washed with a standard shampoo, rinsed again and then dried.

The color was then measured with a MINOLTA CM2002 calorimeter in the L*a*b* system.

In the L*a*b* system, the 3 parameters denote respectively the intensity (L*), the shade (a*) and the saturation (b*).

According to this system, the higher the value of L, the lighter or less intense the color. Conversely, the lower the value of L, the deeper or more intense the color.

The results have been grouped together in Table (I) below.

TABLE I

| EXAMPLE | L* |
|---|---|
| 2 | 26.66 |
| 1 | 22.21 |

Conclusion:

The dyeing with the combination according to the invention (1) is more intense than that of the prior art (2) [lower L value].

What is claimed is:

1. A composition for oxidation dyeing keratinous fibers comprising, in a medium suitable for dyeing:
(i) at least one oxidation dye precursor chosen from 1-(4-aminophenyl)-pyrrolidines of formula (I) and acid addition salts thereof: 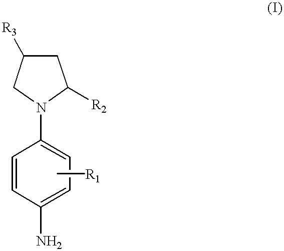
wherein:
R₁ is chosen from a hydrogen atom, C₁-C₆ alkyl groups, C₁-C5 monohydroxyalkyl groups, and C₂-C₅ polyhydroxyalkyl groups,
R₂ is chosen from a hydrogen atom, a —CONH₂ group, C₁-C₅ monohydroxyalkyl groups, and C₂-C₅ polyhydroxyalkyl groups, and
R₃ is chosen from a hydrogen atom, and a hydroxyl group, and
(ii) at least one thickening polymer comprising at least one sugar unit.

2. A composition according to claim 1, wherein said keratinous fibers are human keratinous fibers.

3. A composition according to claim 2, wherein said human keratinous fibers are human hair.

4. A composition according to claim 1, wherein said R₁, said R₂, and said R₃ are each a hydrogen atom.

5. A composition according to claim 1, wherein said R₁ and said R₃ are each a hydrogen atom and said R₂ is a —CH₂OH group.

6. A composition according to claim 1, wherein said R₁ is a hydrogen atom, said R₂ is a —CH₂OH group, and said R₃ is a hydroxyl group.

7. A composition according to claim 1, wherein said R₁ and said R₃ are each a hydrogen atom and said R₂ is a —CONH₂ group.

8. A composition according to claim 1, wherein said acid addition salts of said 1-(4-aminophenyl)pyrrolidines of formula (I) are chosen from hydrochlorides, hydrobromides, sulphates, tartrates, lactates, and acetates.

9. A composition according to claim 1, wherein said at least one oxidation dye precursor is present in said composition in an amount ranging from 0.001% to 10% by weight relative to the total weight of the composition.

10. A composition according to claim 9, wherein said at least one oxidation dye precursor is present in said composition in an amount ranging from 0.01% to 8% by weight relative to the total weight of the composition.

11. A composition according to claim 1, wherein said at least one thickening polymer comprising at least one sugar unit is chosen from:
(1) nonionic guar gums;
(2) biopolysaccharide gums of microbial origin;
(3) gums derived from plant exudates;
(4) pectins;
(5) alginates;
(6) starches; and
(7) hydroxy(C₁-C₆)alkyl celluloses and carboxy(C₁-C₆)alkyl celluloses.

12. A composition according to claim 11, wherein said biopolysaccharide gums of microbial origin are chosen from Scleroglucan gum and Xanthan gum.

13. A composition according to claim 1 1, wherein said gums derived from plant exudates are chosen from gum Arabic, Ghatti gum, Karaya gum, Tragacanth gum, Carrageenan gum, Agar gum, and Carob gum.

14. A composition according to claim 1, wherein said at least one thickening polymer comprising at least one sugar unit is chosen from unmodified nonionic guar gums.

15. A composition according to claim 1 1, wherein said hydroxy(C₁-C₆)alkyl celluloses are chosen from hydroxyethyl celluloses and hydroxypropyl celluloses.

16. A composition according to claim 11, wherein said carboxy(C₁-C₆)alkyl celluloses are chosen from carboxymethyl celluloses.

17. A composition according to claim 1, wherein said at least one thickening polymer comprising at least one sugar unit is present in said composition in an amount ranging from 0.01% to 10% by weight relative to the total weight of said composition.

18. A composition according to claim 1, wherein said at least one thickening polymer comprising at least one sugar unit is present in said composition in an amount ranging from 0.1% to 5% by weight relative to the total weight of said composition.

19. A composition according to claim 1 further comprising at least one coupler.

20. A composition according to claim 19, wherein said at least one coupler is chosen from meta-phenylenediamines, meta-aminophenols, meta-diphenols, naphthols, heterocyclic couplers, and acid addition salts of any of the foregoing compounds.

21. A composition according to claim 19, wherein said at least one coupler is chosen from 2,4-diamino-1-(β-hydroxyethyloxy)benzene, 2-methyl-5-aminophenol, 5-N-(β-hydroxyethyl) amino-2-methylphenol, 3-aminophenol, 1,3-dihydroxybenzene, 1,3-dihydroxy-2-methylbenzene, 4-chloro-1,3-dihydroxy-benzene, 2-amino-4-(β-hydroxyethylamino)-1-methoxy-benzene, 1,3-diaminobenzene, 1,3-bis(2,4-diamino-phenoxy)propane, sesamol, 1-amino-2-methoxy-4,5-methylenedioxybenzene, α-naphthol, 6-hydroxyindole, 4-hydroxyindole, 4-hydroxy-N-methylindole, 6-hydroxy-indoline, 2,6-dihydroxy-4-methylpyridine, 1-H-3-methyl-pyrazol-5-one, 1-phenyl-3-methylpyrazol-5-one, 2-amino-3-hydroxypyridine, 3,6-dimethylpyrazolo[3,2-c]-1 ,2,4-triazole, 2,6-dimethylpyrazolo[1,5-b]-1,2,4-triazole and acid addition salts of any of the foregoing compounds.

22. A composition according to claim 19, wherein said at least one coupler is present in said composition in an amount ranging from 0.0001% to 15% by weight relative to the total weight of the composition.

23. A composition according to claim 1 further comprising at least one oxidation base, other than said at least one oxidation dye precursor, in an amount ranging from 0.0001% to 15% by weight relative to the total weight of said composition.

24. A composition according to claim 1 further comprising at least one direct dye in an amount ranging from 0.001% to 20% by weight relative to the total weight of said composition.

25. A composition according to claim 1 further comprising at least one agent chosen from reducing agents and antioxidants, wherein said at least one agent is present in said composition in an amount ranging from 0.05% to 1.5% by weight relative to the total weight of said composition.

26. A composition according to claim 1 further comprising at least one cationic polymer in an amount of at least 0.01% by weight relative to said composition.

27. A composition for oxidation dyeing keratinous fibers comprising, in a medium suitable for dyeing:
(i) at least one oxidation dye precursor chosen from 1-(4-aminophenyl)-pyrrolidines of formula (I) and acid addition salts thereof: 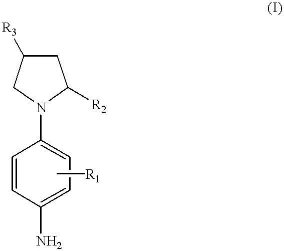
wherein:
R₁ is chosen from a hydrogen atom, C₁-C₅ alkyl groups, C₁-C₅ monohydroxyalkyl groups, and C₂-C₅ polyhydroxyalkyl groups,
R₂ is chosen from a hydrogen atom, a —CONH₂ group, C₁-C₅ monohydroxyalkyl groups, and C₂-C₅ polyhydroxyalkyl groups, and
R₃ is chosen from a hydrogen atom, and a hydroxyl group, and
(ii) at least one thickening polymer comprising at least one sugar unit, and
(iii) at least one oxidizing agent.

28. A composition according to claim 27, wherein said at least one oxidizing agent is chosen from hydrogen peroxide, urea peroxide, alkali metal bromates, alkali metal ferricyanides, persalts, and oxidation-reduction enzymes

29. A composition according to claim 28, wherein said at least one oxidizing agent is chosen from laccases, peroxidases and 2-electron oxidoreductases, where appropriate in the presence of their respective donor or cofactor.

30. A composition according to claim 28, wherein said at least one oxidizing agent is hydrogen peroxide.

31. A composition according to claim 28, wherein said at least one oxidizing agent comprises a solution of hydrogen peroxide with a titre ranging from 1 to 40 in volume.

32. A composition according to claim 1, wherein said composition for oxidation dyeing keratinous fibers has a pH ranging from 3 to 12.

33. A composition according to claim 27, wherein said composition for oxidation dyeing keratinous fibers has a pH ranging from 3 to 12.

34. A composition according to claim 27 further comprising at least one surfactant chosen from anionic surfactants, cationic surfactants, nonionic surfactants, and amphoteric surfactants.

35. A composition according to claim 34, wherein said at least one surfactant is present in said composition in an amount ranging from 0.1% to 20% by weight relative to the total weight of said composition.

36. A method for oxidation dyeing keratinous fibers comprising:
(A) applying to said fibers at least one dyeing composition comprising, in a medium suitable for dyeing:
(i) at least one oxidation dye precursor chosen from 1-(4-aminophenyl)-pyrrolidines of formula (I) and acid addition salts thereof: 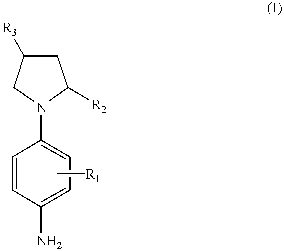
wherein:
R₁ is chosen from a hydrogen atom, C₁-C₆ alkyl groups, C₁-C₅ monohydroxyalkyl groups, and C₂-C₅ polyhydroxyalkyl groups,
R₂ is chosen from a hydrogen atom, a —CONH₂ group, C₁-C₅ monohydroxyalkyl groups, and C₂-C₅ polyhydroxyalkyl groups, and
R₃ is chosen from a hydrogen atom, and a hydroxyl group, and optionally comprising:
(ii) at least one thickening polymer comprising at least one sugar unit, and
(B) developing a color by applying to said fibers at least one oxidizing composition comprising:
at least one oxidizing agent and
optionally comprising said at least one thickening polymer comprising at least one sugar unit,
wherein said at least one oxidizing composition is
applied to said fibers after combining, at the time of use, said at least one oxidizing composition with said at least one dyeing composition, or
applied to said fibers either simultaneously with or immediately after said at least one dyeing composition, without intermediate rinsing, and
(C) provided that said at least one thickening polymer comprising at least one sugar unit is present in at least one of said at least one dyeing composition or said at least one oxidizing composition.

37. A method according to claim 36, wherein said keratinous fibers are human keratinous fibers.

38. A method according to claim 37, wherein said human keratinous fibers are human hair.

39. A method for oxidation dyeing keratinous fibers comprising:
(A) preparing at least one dyeing composition comprising, in a medium suitable for dyeing:
(i) at least one oxidation dye precursor chosen from 1-(4-aminophenyl)-pyrrolidines of formula (I) and acid addition salts thereof: 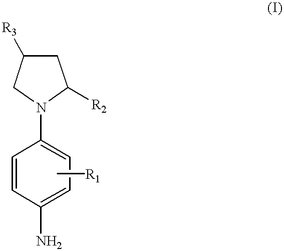
wherein:
R₁ is chosen from a hydrogen atom, C₁-C₆ alkyl groups, C₁-C₅ monohydroxyalkyl groups, and C₂-C₅ polyhydroxyalkyl groups,
R₂ is chosen from a hydrogen atom, a —CONH₂ group, C₁-C₅ monohydroxyalkyl groups, and C₂-C₅ polyhydroxyalkyl groups, and
R₃ is chosen from a hydrogen atom, and a hydroxyl group,
(ii) at least one thickening polymer comprising at least one sugar unit, and
(iii) at least one oxidizing agent,
(B) developing a color by applying said at least one dyeing composition prepared in (A) above to said fibers,
(C) leaving said at least one dyeing composition prepared in (A) above on said fibers for a time ranging from 1 to 60 minutes,
(D) rinsing said fibers, optionally shampooing said fibers, and optionally further rinsing said fibers, and
(E) drying said fibers.

40. A method according to claim 39, wherein said keratinous fibers are human keratinous fibers.

41. A method according to claim 40, wherein said human keratinous fibers are human hair.

42. A method according to claim 39, wherein said leaving time is a time ranging from 10 to 45 minutes.

43. A method according to claim 39, wherein said human hair is chosen from wet human hair and dry human hair.

44. A kit for oxidation dyeing keratinous fibers comprising at least two compartments, wherein:
(A) a first compartment comprises at least one dyeing composition comprising, in a medium suitable for dyeing:
(i) at least one oxidation dye precursor chosen from 1-(4-aminophenyl)-pyrrolidines of formula (I) and acid addition salts thereof: 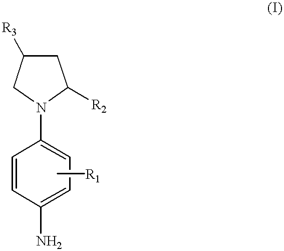
wherein:
R₁ is chosen from a hydrogen atom, C₁-C₆ alkyl groups, C₁-C₅ monohydroxyalkyl groups, and C₂-C₅ polyhydroxyalkyl groups,
R₂ is chosen from a hydrogen atom, a —COONH₂ group, C₁-C₅ monohydroxyalkyl groups, and C₂-C₅ polyhydroxyalkyl groups, and
R₃ is chosen from a hydrogen atom, and a hydroxyl group, and optionally comprising:
(ii) at least one thickening polymer comprising at least one sugar unit,
(B) a second compartment comprises at least one oxidizing agent and optionally comprises said at least one thickening polymer comprising at least one sugar unit, and
(C) provided that said at least at least one thickening polymer comprising at least one sugar unit is present in at least one of said first compartment or said second compartment.

45. A kit according to claim 44, wherein said keratinous fibers are human keratinous fibers.

46. A kit according to claim 45, wherein said human keratinous fibers are human hair.

47. A kit for oxidation dyeing keratinous fibers comprising at least three compartments, wherein:
(A) a first compartment comprises at least one dyeing composition comprising, in a medium suitable for dyeing, at least one oxidation dye precursor chosen from 1-(4-aminophenyl)-pyrrolidines of formula (I) and acid addition salts thereof: 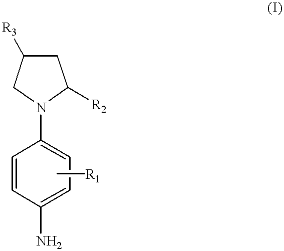
wherein:
R₁ is chosen from a hydrogen atom, C₁-C₆ alkyl groups, C₁-C₅ monohydroxyalkyl groups, and C₂-C₅ polyhydroxyalkyl groups,
R₂ is chosen from a hydrogen atom, a —CONH₂ group, C₁-C₅ monohydroxyalkyl groups, and C₂-C₅ polyhydroxyalkyl groups, and
R₃ is chosen from a hydrogen atom, and a hydroxyl group,
(B) a second compartment comprises at least one thickening polymer comprising at least one sugar unit, and
(C) a third compartment comprises at least one oxidizing agent.

48. A kit according to claim 47, wherein said keratinous fibers are human keratinous fibers.

49. A kit according to claim 48, wherein said human keratinous fibers are human hair.
